(19) United States
(12) Patent Application Publication
(10) Pub. No.: US 2026/0053536 A1
(43) Pub. Date: Feb. 26, 2026

(54) WIRELESSLY OPERABLE IMPLANT SYSTEM FOR PROVIDING CONTROLLED LENGTHENING OF A BONE

(71) Applicant: RAYCURA MEDICAL TECHNOLOGIES PRIVATE LIMITED, Nagpur, Maharashtra (IN)

(72) Inventors: Rohan Milind DESHPANDE, Nagpur (IN); Ayush Sanjay GAIKWAD, Chandkapur (IN)

(73) Assignee: RAYCURA MEDICAL TECHNOLOGIES PRIVATE LIMITED, Nagpur, Maharashtra (IN)

(21) Appl. No.: 19/105,354

(22) PCT Filed: Aug. 22, 2023
(86) PCT No.: PCT/IN2023/050792

§ 371 Date: Feb. 21, 2025

(30) Foreign Application Priority Data
Aug. 23, 2022 (IN) 202221048016

(51) Int. Cl. A61B 17/72; A61B 17/00

(52) U.S. Cl. CPC A61B 17/7225; A61B 2017/00075; A61B 2017/00221; A61B 2017/00398

(57) ABSTRACT

The present invention discloses a wirelessly operable implant system for providing controlled lengthening of a bone. The wirelessly operable implant system (100) comprises a bone lengthening intramedullary nail (110), an extracorporeal wireless controller (130), and an implantable drive unit (120). The bone lengthening intramedullary nail (110) fixes in intramedullary cavity (16) and its extending elements (111) and (112) fix with different bone segments (14) and (15). The bone lengthening intramedullary nail (110) distracts the bone segments (14) and (15) when provided with input rotational motion to the unit (113) fitted within. This input rotational motion is given with physical connection by extramedullary implant (120) which is intracorporal and extramedullary. The extramedullary implant (120) is operated by an extracorporeal wireless controller (130).

### FIELD OF INVENTION

The present invention in general relates to the field of biomedical engineering and more particularly to a wirelessly operable implant system for providing controlled lengthening of a bone.

### BACKGROUND OF THE INVENTION

Distraction osteogenesis is a technique used in orthopedic surgery to repair skeletal deformities, reconstruction surgery, and to treat fractures. Limb lengthening surgery is one such technique that is used to lengthen (increase the length) a bone (for example a femur or tibia). This technique increases the length of the bone ultimately increasing the length of the limb (as muscles, fascia, veins nerves, etc. all stretch and lengthen in the process). In medical conditions with skeletal defects such as short stature, leg length discrepancy, pseudoarthrosis, bone infection, bone cancer, malunion, nonunion of bone and prior bone fracture did not heal correctly while treating fractures, etc. due to congenital or acquired reasons, can be treated with distraction osteogenesis. Bone lengthening nails also called distraction rods or distraction implants and external frames called external fixators and Ilizarov frames are devices used in the process of distraction osteogenesis for limb lengthening and reconstruction surgeries. These devices are also useful for bone compression. Existing solutions such as external fixators are cumbersome for patients and prone to infection which requires additional surgeries. Hence intramedullary bone lengthening nail prefers over external fixators as the intramedullary nail is fitted inside the intramedullary cavity of the bone, it drastically reduces the chance of infection and is also comfortable for patients as nothing comes out of the body.

Although various attempts are made for providing various means for bone lengthening and few of them are discussed below.

EP1613226B1 discloses a device which lengthens bones or bone parts, especially for segmental transportation, comprising at least two elements which can be displaced in relation to each other. At least one locking element can be axially displaced in or along a guiding element.

U.S. Pat. No. 9,408,644B2 discloses a length-adjustable IM nail system includes a telescoping IM nail with proximal and distal bodies. An inner magnet within the proximal body is connected to a threaded rod which, in turn, is connected to the distal body. The threaded rod passes through a threaded block which is connected to the proximal body. The position of the distal end of the threaded rod is fixed with respect to the distal body, but may rotate freely within this fixed position. An actuator is also disclosed that includes a pair of rotating magnets disposed in an angular relationship with each other and the axis of the IM nail and the patient's limb. Rotation of these outer magnets in the same direction results in rotation of the inner magnet and threaded rod and a telescoping axial movement of the threaded block and proximal body with respect to the distal body.

The technology disclosed in the above arts and some intramedullary nails based upon it have various limitations. These intramedullary nails have various distraction mechanisms in it along with electronics and a drive system (sometimes motor) placed inside it. All these components are cramped in the small form factor of an intramedullary nail. To accommodate all these components in a small space inside the nail, the structural strength of the intramedullary nail gets compromised and the functions of electronics to be employed also get constrained. This reduces the reliability and efficacy of the intramedullary bone lengthening nails. The existing Bone lengthening nails also can't provide full weight-bearing capacity to patients due to lack of strength, which ultimately makes patients dependent on crutches or walkers for walking and daily activity during the lengthening phase of 1 to 8 months at least. The strength and functionality of these bone-lengthening nails can be made better at the cost of a bigger form factor (size) which makes it difficult to be implanted in the first place, due to space constraints of the intramedullary canal.

Therefore, there is a need for a wirelessly operable implant system for providing controlled lengthening of a bone to overcome the drawbacks of existing intramedullary bone lengthening nail technologies.

### OBJECT OF THE INVENTION

The primary object of the present invention is to provide a wirelessly operable implant System for providing controlled lengthening of a bone.

Another object of the present invention is to provide bone lengthening intramedullary nail that is compact yet sturdy and reliable, without fragile components.

Yet another object of the present invention is to provide a system to operate a bone-lengthening intramedullary nail wirelessly by an extracorporeal wireless controller.

Other objects, features, and advantages will become apparent from detailed descriptions and appended claims to those skilled in the art.

### SUMMARY OF THE INVENTION

An embodiment of the present invention discloses a wirelessly operable implant system (100) for providing controlled lengthening of a bone. The wirelessly operable implant system (100) comprising an intramedullary bone lengthening nail (110) attached to bone segments (14, 15), wherein the intramedullary bone lengthening nail (110) configured to one of distract bone segments (14, 15) and compress bone segments (14, 15) upon receiving a rotational motion in one of a clockwise direction and anticlockwise direction, at an input member (114) of a motion translation unit (113), the motion translation unit (113) is integrated in the bone lengthening intramedullary nail (110); an extramedullary implant (120) operatively connected to the intramedullary bone lengthening nail (110) for providing the rotational motion in one of the clockwise direction and anticlockwise direction, at the input member (114) of the motion translation unit (113), the extramedullary implant (120) comprises a receiver (121) configured to receive at least one of energy and one or more control signals wirelessly; an implant control circuit (122); an actuator 123 configured to provide one or more actuations for providing the rotational motion to the input member (114) of the motion translation unit (113) whereby operating the bone lengthening intramedullary nail (110); one or more actuation feedback sensors configured to sense the provided one or more actuations; acoustic transmitter configured to transmit one or more acoustic signals wirelessly; and an extracorporeal wireless controller (130) operatively connected to the extramedullary implant (120) for transmitting at least one of the energy and one or more control signals, and receiving the one or more acoustic signals, the extracorporeal wireless controller (130) comprises a transmitter (132) configured for transmitting least one of the energy and one or more control signals wirelessly to the extramedullary implant (120); a control board (131); and an acoustic receiver (133) configured for receiving an acoustic signal as a feedback signal from the said extramedullary implant (120), whereby providing the controlled lengthening of the bone.

In one embodiment of the present invention, the extracorporeal wireless controller system (130), comprises: a transmitter (132) configured to generate electromagnetic waves modulated in patterns corresponding to distinct control signals of the control signals, wherein the electromagnetic waves transfer the energy and one or more control signals concurrently; and a receiver (121) of the extramedullary implant (120) configured to receive at least one of the energy and the one or more control signals, wherein a control circuit (122) of the extramedullary implant (120) is configured to interpret the said signals to execute one or more actions including one of lengthening actuation, compression actuation, transmitting identity, and transmitting actuation feedback.

In another embodiment of the present invention, the extramedullary implant (120) configured to perform a bidirectional exchange of wireless signals with the extracorporeal wireless controller (130), the extramedullary implant (120) configured to transmit the one or more acoustic signals including identity details, actuation performed by actuator (123), and status of the extramedullary implant (120), to the extracorporeal wireless controller (130), wherein the extracorporeal wireless controller (130) upon receiving the acoustic signal from the extramedullary implant (120), configured to determine one or more course of actions, the one or more course of actions comprises one of transmitting at least one of the energy, and one or more control signals corresponding to lengthening of bone, compression of bone and query for identity of the extramedullary implant (120); and interrupting transmitting of at least one of the energy and signal.

Another embodiment of the present invention discloses a wirelessly operable implant system (100) for providing controlled lengthening of a bone comprising an intramedullary bone lengthening nail (110) attached to bone segments, wherein the intramedullary bone lengthening nail (110) configured to one of distract bone segments (14, 15) and compress bone segments (14, 15) upon receiving rotational motion in one of a clockwise direction and anticlockwise direction, at an input member (114) of a motion translation unit (113); an extramedullary implant (140) operatively connected to the intramedullary bone lengthening nail (110) for providing the rotational motion in one of the clockwise direction and anticlockwise direction, at the input member (114) of the motion translation unit (113), wherein the extramedullary implant (140) configured to be operated magnetically, comprises a driven magnetic array (141) coupled to a gearbox (142), wherein the gearbox (142) operatively connected to a connecting shaft (115), the connecting shaft (115) operatively connected to the input member (114) of the motion translation unit (113); and an extracorporeal wireless controller (130) operatively connected to the extramedullary implant (140) for magnetically operating the extramedullary implant (140), the extracorporeal wireless controller (130) comprises a control board (131); a driver magnetic array (135); and a display (134), the extracorporeal wireless controller (130) is configured to be placed on a skin extracorporeally (111) covering the magnetically operated the extramedullary implant (140), whereby the driver magnetic array (135) is magnetically coupled to the driven magnetic array (141) for providing the controlled lengthening of the bone.

In one embodiment of the present invention, the extracorporeal wireless controller (130) is configured to be controlled based on one or more inputs received from a user through the control board (131) to rotate the driver magnetic array (135) in a designated direction.

In another embodiment of the present invention, the driven magnetic array (141) is configured to synchronize with a rotation of the driver magnetic array (135), the rotation of the driven magnetic array (141) is configured to transmit motion to the gearbox (142) attached to the connecting shaft (115).

In yet another embodiment of the present invention, the bone lengthening intramedullary nail (110) is integrated with a motion translation unit (113).

In further embodiment of the present invention, the gearbox (142) is configured to provide torque multiplication to drive the input member (114) of the motion translation unit (113), whereby the motion translation unit (113) configured to transmit the rotational motion to the input member of motion translation unit of the bone lengthening intramedullary nail (110), for providing the controlled lengthening of the bone.

Since the extramedullary implant is positioned above the bone, it is subject to fewer limitations in terms of its size and configuration compared to intramedullary implantation. The additional space provided by the extramedullary implantation offers the opportunity to incorporate more safety features, thus enhancing the overall reliability of the system. An advantage of the extramedullary approach is its improved safety aspect. In a particular situation where the extramedullary implant requires removal or replacement, it is possible to accomplish this through a minor surgical procedure. Importantly, this procedure will not interfere with the bone fixation that has been achieved through the use of the intramedullary nail for bone lengthening. This contrasts with existing technologies where this level of intervention is not feasible. Moreover, following the completion of the bone distraction phase that spans over several months, the removable extramedullary implant can be effortlessly extracted from the body, leaving no electronic components of the present invention within the body. This feature is attributed to its separable nature from the bone lengthening intramedullary device.

### BRIEF DESCRIPTION OF DRAWINGS

This invention is described by way of example with reference to the following drawings. These drawings being referred herein are for the purpose of illustrating preferred embodiments of the invention only, and not for the purpose of limiting the same.

FIG. 1 illustrates a deployed wirelessly operable implant system for providing controlled lengthening of a bone, according to an embodiment of the present invention.

FIG. 2 illustrates a wirelessly operable implant system for providing controlled lengthening of a bone and a flow diagram of communication between an extracorporeal wireless controller and an extramedullary implant, according to an embodiment of the present invention.

FIG. 3 illustrates a deployed wirelessly operable implant system for providing controlled lengthening of a bone, according to another embodiment of the present invention.

### DETAILED DESCRIPTION OF THE ACCOMPANYING DRAWINGS

The present invention is described hereinafter by various embodiments with reference to the accompanying drawings, wherein reference numerals used in the accompanying drawings correspond to the like elements throughout the description. This invention may, however, be embodied in many different forms and should not be construed as limited to the embodiments set forth herein. Rather, the embodiments are provided so that this disclosure will be thorough and complete and will fully convey the scope of the invention to those skilled in the art. In the following description, numeric values and ranges are provided for various aspects of the implementations described. These values and ranges are to be treated as examples only and are not intended to limit the scope of the invention. In addition, a number of materials are identified as suitable for various facets of the implementations. These materials are to be treated as exemplary and are not intended to limit the scope of the invention.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, Appearances of the phrase “in an embodiment”, “in another embodiment” and similar language throughout this specification may, but not necessarily do, all refer to the same embodiment. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein are solely used for descriptive purposes and should not be construed as limiting in scope.

The systems, methods, and examples provided herein are only illustrative and not intended to be limiting. The present invention relating to the Wirelessly Operable Implant System for controlled lengthening of Bone is a new concept that can be understood by way of the following embodiments below:

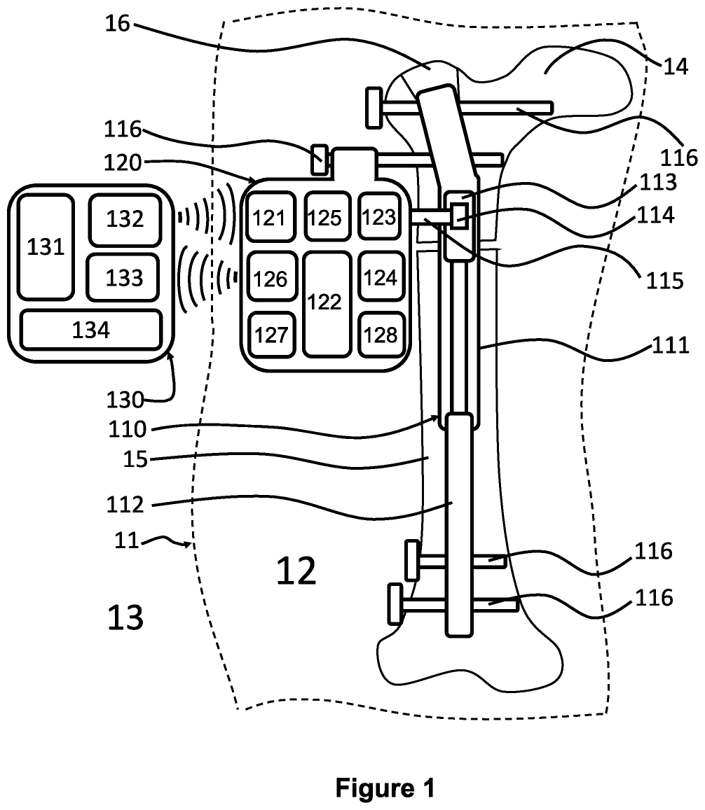

FIG. 1 illustrates a deployed wirelessly operable implant system for providing controlled lengthening of a bone, according to an embodiment of the present invention.

The wirelessly operable implant system (100) for providing controlled lengthening of a bone comprises a bone lengthening (distracting) intramedullary nail (110), an extracorporeal wireless controller (130), and an extramedullary implant (120).

A bone-lengthening intramedullary nail (110) is fitted inside an intramedullary cavity (16), wherein a housing (111) of the bone lengthening intramedullary nail (110) fixes to a first bone segment (14) and another extending part an extender (112) of the bone lengthening intramedullary nail (110) fixes to a second bone segment (15). The bone lengthening intramedullary nail (110) has a motion translation unit (113) fitted inside it, wherein rotation of an input member (114) of the motion translation unit (113) in one direction drives the motion translation unit (113) resulting in sliding of the extender (112) connected to the second bone segment (15) relative to the housing (111) connected to the first bone segment (14) varying gap between those bone segments (14), and (15) by rotating the input member (114) in another direction of rotation the direction of liner displacement (sliding) of the extender (112) changes (reverses). The bone lengthening intramedullary nail (110) has a complete mechanical operation. The input member's (114) rotation axis is nonparallel (preferably perpendicular) to the direction of linear displacement of the extender (112). Radially to the bone segments (14) and (15) and the bone lengthening intramedullary nail (110).

The extracorporeal wireless controller (130) mainly comprises a transmitter (132), a control board (131) and a display (134). Wherein according to the function selected by the user on the control board (131), the transmitter (132) transmits energy and signal wirelessly. The signals that are transmitted signify different actions to be taken that include but are not limited to the initialization signal to get device identity, the lengthening signal, and the compression signal.

The extracorporeal wireless controller (130) with its transmitter (132), transmits the energy and signal concurrently to the extramedullary implant's (120) receiver (121). This is done by the transmitter (132) transmitting the energy in modulated patterns, where each pattern of transmission signifies (signals) actions to be taken by the extramedullary implant (120). The energy and signal are preferably sent by electromagnetic waves. As the electromagnetic waves can transfer both the energy and signals. By modulating the energy-transferring electromagnetic waves in different patterns (wherein different modulated patterns are different signals corresponding to different actions to be taken by the extramedullary implant (120)) the control signals and the energy can both be transmitted to the implanted extramedullary implant (120) concurrently. And the implanted extramedullary implant (120) can receive both the energy and signal concurrently with the same hardware without requiring additional hardware to be implanted in the body.

The extracorporeal wireless controller (130) also has an acoustic receiver (133) to receive feedback from the implanted extramedullary implant (120).

An extramedullary implant (120) of this embodiment comprises a receiver (121), an implant control circuit (122), an actuator (123), a feedback sensor (124), an indicator (125), an acoustic transmitter (126), an on board energy storage (127), and a drain (128).

The electromagnetic waves transmitted by the transmitter (132) of an extracorporeal wireless controller (130) are received by the receiver (121) and are further converted to electrical energy; the same electrical energy is used for the extramedullary implant's (120) operations and is also stored in trivial quantity for a brief amount of time, by the on-board energy storage (127). The implant control circuit (122) also extracts the signal from the same electromagnetic waves by analyzing its modulated pattern. The implant control circuit (122) further processes the signal and functions accordingly. The signal has encoded meanings defining the actuation direction of rotation, number of rotations, and speed of actuation, or identity confirmation signal. Also, the signal is used to check the proper positioning of transmitter (132) over the receiver (121).

The actuator (123) of the extramedullary implant (120) is an electrically powered actuator that converts electrical energy into rotational motion. The actuator (123) can have an integrated gearbox coupled to reduce speed and multiply torque. The connecting shaft (115) is attached at the actuator end to transfer the actuator's (123) rotational motion to the input member of the motion translation unit (114) of the bone lengthening intramedullary nail (110). The connecting shaft (115) is designed to operatively couple with the input member (114) of the motion translation unit (113) of the bone lengthening intramedullary nail (110) to provide it the rotation for varying the distance between bone segments (14, 15).

The extramedullary implant (120) itself is mounted over the first bone (14) (extramedullary) inside the body (is Intracorporal 12) via a bone screw (116).

The feedback sensor (124) senses the parameters of actuation being performed and gives it to the implant control circuit (122).

The acoustic transmitter (126) is used to transmit the actuation feedback to the extracorporeal wireless controller (130). The acoustic transmitter (126) is also used to transmit a signal to the extracorporeal wireless controller (130) to confirm its proper positioning relative to the extracorporeal wireless controller's (130) transmitter (132). The feedback of actuation is also indicated by an indicator (125). The Indicator (125) is preferably giving haptic feedback.

The excess energy available in the on-board energy storage (127) can be used up by the drain 128 in the circuit if required.

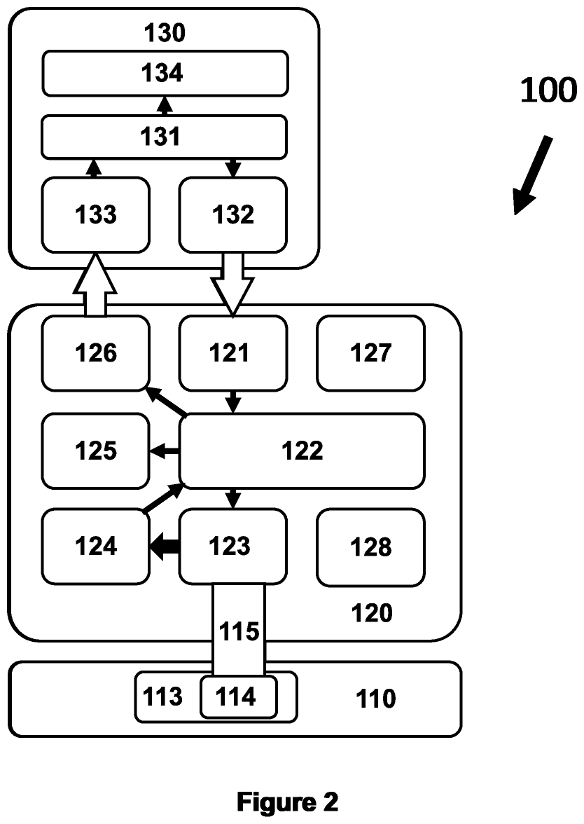

FIG. 2 illustrates a wirelessly operable implant system for providing controlled lengthening of a bone and a flow diagram of communication between an extracorporeal wireless controller and an extramedullary implant, according to an embodiment of the present invention.

According to an embodiment of the present invention, the steps of working of the system comprises:

At step 1, the extracorporeal wireless controller (130) transmits the energy and signal with the transmitter (132) as described above wherein firstly initialization signal is sent.

At step 2, the extramedullary implant's (120) receiver (121) receives the energy and turns on the extramedullary implant 120, then according to the signal being received it transmits its identity back to the extracorporeal wireless controller (130). This is done by an acoustic transmitter (126), wherein the signal is acoustically modulated. The signal primarily sends identity information like the bone segment it is connected to (tibia, femur, humerus) and the side of the body (Left, right) and proper positioning of transmitter over the receiver (121), but is not limited to the same.

At step 3, the acoustic receiver (133) receives the signal sent by the Extramedullary implant (120). The regular practice of limb lengthening suggests distracting bone at a rate of 1 mm per day, however, it varies according to the patient, and the doctor sets the routine accordingly. To avoid excess lengthening by patients, the Extracorporeal wireless controller's (130) is equipped with a clock and a data logger in its control board (131) to store the information of lengthening is performed. It also stores the net lengthening performed over the period to ensure no excess lengthening is performed.

At step 4, the extracorporeal wireless controller's (130) control board (131) confirms with previous data (logged data) that lengthening for the day and net lengthening is yet to be achieved. After confirming both conditions and confirming that lengthening is to be done the control board (131) sends the energy with integrated lengthening signal via the transmitter (132).

At step 5, the extramedullary implant's (120) receiver (121) receives the energy and lengthening signal and further passes it to implant control circuit (122). The implant control circuit (122) gets powered up analyses the signal and directs actuator (123) to perform the rotational actuation accordingly which further drives the operatively coupled input member of the motion translation unit (114) of the bone lengthening intramedullary nail (110) distracting the bone segments (14,15) as required. After a certain amount of rotation is performed by the actuator (123) (for example the actuator turns 6 degrees) the feedback sensor (124) sends feedback to the implant control circuit (122) which further transmits that information to the Extracorporeal wireless controller (130) acoustically via the acoustic transmitter (126) signaling estimated lengthening performed.

At step 6, after receiving the acoustic feedback from the acoustic receiver (133), the extracorporeal wireless controller's (130) control board (131) checks back to step four and continues the process until lengthening for the set lengthening or net lengthening, or any other set condition is achieved.

At step 7, in some cases, the doctor needs to compress the bone segments (14,15) which means reducing the gap in the bone segments (14, 15), in such scenarios the Extracorporeal wireless controller (130) is set to send the energy and compression signal to the Extramedullary implant (120) to the drive actuator (123) in reverse rotational direction that further does bone compression.

According to an embodiment of the present invention, the transmitter (132) of the extracorporeal wireless controller (130) and the receiver (121) of the extramedullary implant (120) can also be one of inductive coupling, acoustic coupling, ultrasonic coupling, pressure coupling, optical coupling, vibration coupling, and magnetic coupling.

According to an embodiment of the present invention, the acoustic transmitter (126) of the extramedullary implant (120) and the acoustic receiver (133) of the extracorporeal wireless controller (130) can be replaced with an inductive coupling, ultrasonic coupling, pressure coupling, optical coupling, vibration coupling, and magnetic coupling.

According to an embodiment of the present invention, the energy storage (127) of enhanced capacity can be used to the power extramedullary implant (120) to reduce or eliminate extramedullary implant's (120) reliance on extracorporeal wireless controller (130) for receiving the energy to power itself.

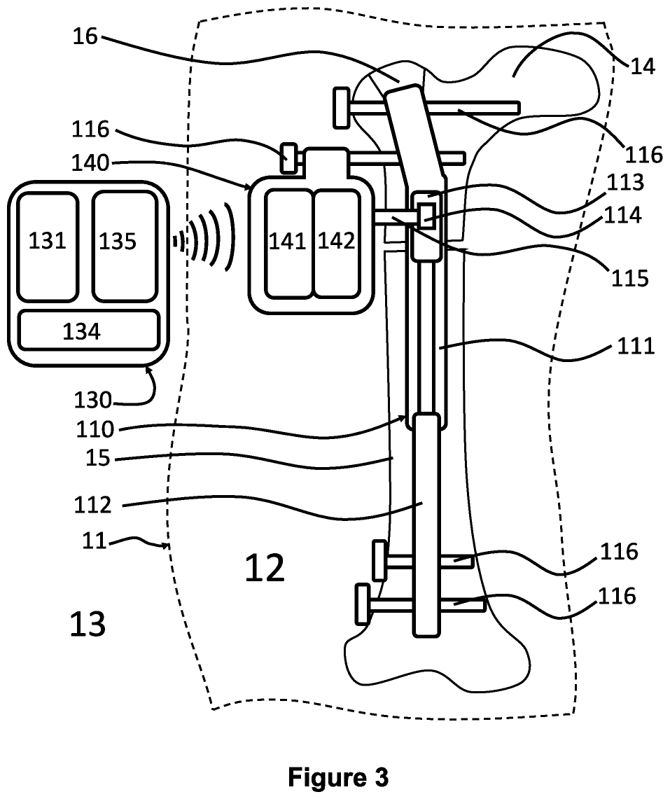

FIG. 3 illustrates a deployed wirelessly operable implant system for providing controlled lengthening of a bone, according to another embodiment of the present invention. A communication between the extracorporeal wireless controller (130) and the magnetically operated extramedullary implant (140) is disclosed. The magnetically operated extramedullary implant (140) has a driven magnetic array (141) that is further connected with a gearbox (142) that is connected to the connecting shaft (115) that further connects to the input member (114) of a motion translation unit (113).

The extracorporeal wireless controller (130) of this embodiment has a driver magnetic array (135) that is rotatable. When the bone lengthening intramedullary nail (110) is to be operated by rotating the input member (114) of the motion translation unit (113). The extracorporeal wireless controller (130) is put on top of a skin (11) over the magnetically operated extramedullary implant (140) such that the driving magnetic array (135) gets operatively coupled to the driven magnetic array (141) to transmit rotational motion. The extracorporeal wireless controller (130) is commanded by the user using the control board (131). The control board (131) operates the driver magnetic array (135) as per the given instruction. The driving magnetic array (135) rotates in the required direction. The driven magnetic array (141) rotates by following the driving magnetic array (135). The rotating driven magnetic array (141) drives gearbox (142) that after torque multiplication drives the input member (114) of the motion translation unit (113) to operate the bone lengthening intramedullary nail (110).

The foregoing description describes five embodiments of the present invention. It should be appreciated that these embodiments are described for the purpose of illustration only, and that numerous alterations and modifications may be practiced by those skilled in the art without departing from the spirit and scope of the invention. It is intended that all such modifications and alterations be included in so far as they come within the scope of the invention as claimed or the equivalents thereof.

Reference Numerals

| Number | Denotes |
|---|---|
| 11 | Skin |
| 12 | Intracorporal region |
| 13 | Extracorporeal region |
| 14 | First Bone segment |
| 15 | Second Bone segment |
| 16 | Intramedullary cavity |
| 100 | Wirelessly operable implant |
| | system |
| 110 | Bone lengthening intramedullary |
| | nail |
| 111 | Housing |
| 112 | Extender |
| 113 | Motion translation unit |
| 114 | Input member of motion |
| | translation unit |
| 115 | Connecting shaft |
| 116 | Bone screw |
| 120 | Extramedullary implant |
| 121 | receiver |
| 122 | Implant control circuit |
| 123 | Actuator |
| 124 | Feedback Sensor |
| 125 | Indicator |
| 126 | Acoustic Transmitter |
| 127 | Energy storage |
| 128 | Drain |
| 130 | Extracorporeal wireless |
| | controller |
| 131 | Control Board |
| 132 | Transmitter |
| 133 | Acoustic Receiver |
| 134 | Display |
| 135 | Driver magnetic array |
| 140 | Magnetically operated |
| | extramedullary implant |
| 141 | Driven magnetic array |
| 142 | Gearbox |

I/We claim:

1. A wirelessly operable implant system (100) for providing controlled lengthening of a bone comprising:
an intramedullary bone lengthening nail (110) attached to bone segments (14, 15), wherein the intramedullary bone lengthening nail (110) configured to one of distract bone segments (14, 15) and compress bone segments (14, 15) upon receiving a rotational motion in one of a clockwise direction and anticlockwise direction, at an input member (114) of a motion translation unit (113),
the motion translation unit (113) is integrated in the bone lengthening intramedullary nail (110);
an extramedullary implant (120) operatively connected to the intramedullary bone lengthening nail (110) for providing the rotational motion in one of the clockwise direction and anticlockwise direction, at the input member (114) of the motion translation unit (113), the extramedullary implant (120) comprises
a receiver (121) configured to receive at least one of energy and one or more control signals wirelessly;
an implant control circuit (122);
an actuator 123 configured to provide one or more actuations for providing the rotational motion to the input member (114) of the motion translation unit (113) whereby operating the bone lengthening intramedullary nail (110);
one or more actuation feedback sensors configured to sense the provided one or more actuations;
acoustic transmitter configured to transmit one or more acoustic signals wirelessly; and
an extracorporeal wireless controller (130) operatively connected to the extramedullary implant (120) for transmitting at least one of the energy and one or more control signals, and receiving the one or more acoustic signals, the extracorporeal wireless controller (130) comprises
a transmitter (132) configured for transmitting least one of the energy and one or more control signals wirelessly to the extramedullary implant (120);
a control board (131); and
an acoustic receiver (133) configured for receiving an acoustic signal as a feedback signal from the said extramedullary implant (120),
whereby providing the controlled lengthening of the bone.

2. The wirelessly operable implant system (100) as claimed in claim 1, wherein the extracorporeal wireless controller system (130), comprises:
a transmitter (132) configured to generate electromagnetic waves modulated in patterns corresponding to distinct control signals of the control signals, wherein the electromagnetic waves transfer the energy and one or more control signals concurrently; and
a receiver (121) of the extramedullary implant (120) configured to receive at least one of the energy and the one or more control signals, wherein a control circuit (122) of the extramedullary implant (120) is configured to interpret the said signals to execute one or more actions including one of lengthening actuation, compression actuation, transmitting identity, and transmitting actuation feedback.

3. The wirelessly operable implant system (100) as claimed in claim 1, wherein the extramedullary implant (120) configured to perform a bidirectional exchange of wireless signals with the extracorporeal wireless controller (130),
the extramedullary implant (120) configured to transmit the one or more acoustic signals including identity details, actuation performed by actuator (123), and status of the extramedullary implant (120), to the extracorporeal wireless controller (130),
wherein the extracorporeal wireless controller (130) upon receiving the acoustic signal from the extramedullary implant (120), configured to determine one or more course of actions,
the one or more course of actions comprises one of
transmitting at least one of the energy, and one or more control signals corresponding to lengthening of bone, compression of bone and query for identity of the extramedullary implant (120); and
interrupting transmission of at least one of the energy and signal.

4. A wirelessly operable implant system (100) for providing controlled lengthening of a bone comprising:
an intramedullary bone lengthening nail (110) attached to bone segments, wherein the intramedullary bone lengthening nail (110) configured to one of distract bone segments (14, 15) and compress bone segments (14, 15) upon receiving rotational motion in one of a clockwise direction and anticlockwise direction, at an input member (114) of a motion translation unit (113);
an extramedullary implant (140) operatively connected to the intramedullary bone lengthening nail (110) for providing the rotational motion in one of the clockwise direction and anticlockwise direction, at the input member (114) of the motion translation unit (113), wherein the extramedullary implant (140) configured to be operated magnetically, comprises
a driven magnetic array (141) coupled to a gearbox (142),
wherein the gearbox (142) operatively connected to a connecting shaft (115),
the connecting shaft (115) operatively connected to the input member (114) of the motion translation unit (113); and
an extracorporeal wireless controller (130) operatively connected to the extramedullary implant (140) for magnetically operating the extramedullary implant (140), the extracorporeal wireless controller (130) comprises
a control board (131);
a driver magnetic array (135); and
a display (134),
the extracorporeal wireless controller (130) is configured to be placed on a skin extracorporeally (111) covering the magnetically operated the extramedullary implant (140), whereby the driver magnetic array (135) is magnetically coupled to the driven magnetic array (141) for providing the controlled lengthening of the bone.

5. The wirelessly operable implant system (100) as claimed in claim 4, wherein the extracorporeal wireless controller (130) is configured to be controlled based on one or more inputs received from a user through the control board (131) to rotate the driver magnetic array (135) in a designated direction.

6. The wirelessly operable implant system (100) as claimed in claim 4, wherein the driven magnetic array (141) is configured to synchronize with a rotation of the driver magnetic array (135),
the rotation of the driven magnetic array (141) is configured to transmit motion to the gearbox (142) attached to the connecting shaft (115).

7. The wirelessly operable implant system (100) as claimed in claim 4, wherein the bone lengthening intramedullary nail (110) is integrated with a motion translation unit (113).

8. The wirelessly operable implant system (100) as claimed in claim 4, wherein the gearbox (142) is configured to provide torque multiplication to drive the input member (114) of the motion translation unit (113),
whereby the motion translation unit (113) configured to transmit the rotational motion to the input member of motion translation unit of the bone lengthening intramedullary nail (110), for providing the controlled lengthening of the bone.
